(19) United States
(12) Patent Application Publication
(10) Pub. No.: US 2023/0363920 A1
(43) Pub. Date: Nov. 16, 2023

(54) UNIVERSAL TIBIAL INSERT WITH ENHANCED MEDIAL CONGRUENCE

(71) Applicant: Joint Development, LLC, Salt Lake City, UT (US)

(72) Inventor: Dermott J. McHugh, Salt Lake City, UT (US)

(21) Appl. No.: 18/198,240

(22) Filed: May 16, 2023

Related U.S. Application Data
(60) Provisional application No. 63/342,536, filed on May 16, 2022.

(51) Int. Cl. A61F 2/38; A61F 2/30

(52) U.S. Cl. CPC A61F 2/389; A61F 2/30734; A61F 2002/30708; A61F 2002/3863; A61F 2002/30112; A61F 2002/30617; A61F 2002/30494; A61F 2002/30448

(57) ABSTRACT

The universal tibial insert includes a keyed lock plate generally symmetrical across a median plane and a frontal plane and is selectively engageable with an asymmetrical tibial base plate in a first orientation and a second orientation approximately 180° relative to the first orientation. This allows an upwardly presented articular surface to reciprocally interface with a medial condyle and a lateral condyle of a femoral component in articulatory relation therewith when the universal tibial insert is in either of the first orientation or the second orientation.

### BACKGROUND OF THE INVENTION

The present invention generally relates to a tibial insert. More specifically, the present invention is directed to a universal tibial insert having an articular surface geometry that remains substantially congruent with a medial condyle of a femoral component when used as part of a right or left total knee replacement system enabled by a universal coupling symmetric across a median and a frontal symmetry plane.

During knee arthroplasty, a femoral component is affixed to the distal end of a patient femur, a tibial baseplate is affixed to the proximal end of a patient tibia, and a tibial insert is positioned between the femoral component and the tibial baseplate. The tibial insert serves as an articular surface about which the femoral component translates when flexing or extending the knee. The tibial insert also helps constrain the femoral component, and specifically with respect to preventing the femoral component from sliding off the tibial baseplate. One goal of a total knee replacement is to mimic the normal kinematics of a healthy knee joint. Generally, during normal flexion of a healthy knee joint, the medial condyle at the distal end of the femur is congruent with one articular surface of the tibial insert, while the motion of the lateral condyle includes translational movement relative to another articular surface of the tibial insert and rotational movement relative to the medial condyle. As such, the surface geometry of the medial and lateral articular surfaces of the tibial insert differ to account for these differences in natural movement. Furthermore, as the anatomy of a patient knee joint differs from person to person, femoral components, tibial baseplates, and tibia inserts may vary in size and shape. Although, manufacturing costs, inventory, and sterilization costs are generally always twofold since two sets of each component are typically needed, namely one set specifically configured for a right total knee replacement and a second set configured for a left total knee replacement.

One example of a prior art medial pivot knee prothesis that provides closer approximation to normal kinematics during knee flexion is shown and described, e.g., in U.S. Pat. No. 6,013,103 to Kaufman, the contents of which are herein incorporated by reference in its entirety. While existing (newer) prior art designs have improved the congruence for the medial condyle and articular surface, the drawback still is that such designs are inefficient from a manufacturing and inventory standpoint because the tibial insert and/or the tibial baseplate are directional, i.e., each manufactured tibial insert and/or tibial baseplate can only be implanted in either the right knee or the left knee; a single conventional tibial insert and/or a single conventional tibial baseplate cannot be implanted in either of the right knee or the left knee. As such, in addition to two femoral components, two tibial inserts and two tibial baseplates still must still be manufactured to accommodate left and right knee arthroplasties. Naturally as a result of these parts not being universally compatible with both the right knee and left knee, the part count is twofold and, as mentioned above, this increases manufacturing complexity and cost, and requires storing upwards of twice as much inventory.

Another undesirable drawback is the potential for an incorrectly sided implant to be used in surgery, which would result in the need for a revision surgery to replace the incorrect implant. This obviously undesirably increases the intrusiveness, time, and cost required to complete the surgery in the event of a mistake.

There exists, therefore, a significant need in the art for a tibial insert universally couplable with a right or a left knee baseplate, such as by way of a base symmetric across an interior and a posterior symmetry plane, and of which includes an articular surface that remains substantially congruent with a medial condyle of a femoral component regardless whether the universal tibial insert is used as part of a right or left total knee replacement system. The present invention fulfills these needs and provides further related advantages.

### SUMMARY OF THE INVENTION

In one embodiment disclosed herein, a universal tibial insert includes a base selectively engageable with a tibial baseplate and an upwardly presented articular surface having a first side geometry and a second side geometry configured to interface with one of a medial condyle or a lateral condyle of a femoral component in articulatory relation thereof. Here, the articular surface is symmetrical about a median plane such that the universal tibial insert may be oriented relative to the tibial baseplate in one of two configurations 180 degrees relative to the other. Specifically, e.g., the first side geometry is relatively more congruent with the medial condyle and the second side geometry is relatively less congruent with the lateral condyle when the universal tibial insert is in a first orientation for use in one of a right total knee arthroplasty or a left total knee arthroplasty. Conversely, the first side geometry is relatively less congruent with the lateral condyle and the second side geometry is relatively more congruent with the medial condyle when the universal tibial insert is in a second orientation for use in the other of the right total knee arthroplasty or the left total knee arthroplasty.

One feature of this embodiment is that the first side geometry and the second side geometry may be reciprocal, namely that the first side geometry and the second side geometry have approximately the same reciprocal congruence relative to either of the medial condyle or the lateral condyle, depending on the orientation. Specifically, e.g., a radii of the medial condyle of the femoral component relative to a radii of the first side geometry, when the universal tibial insert is in the first orientation, or a radii of the second side geometry, when the universal tibial insert is in the second orientation, may be a medial ratio of about 1:1 to 1:1.2. Moreover, a radii of the lateral condyle of the femoral component relative to a radii of the first side geometry, when the universal tibial insert is in the second orientation, or a radii of the second side geometry, when the universal tibial insert is in the first orientation, may be a lateral ratio of about 1:1.1 to 1:2. In the latter embodiment, the relative congruence may result in an anterior gap and/or a posterior gap between the articular surface and the lateral condyle.

The universal tibial insert as disclosed herein is usable with either of a symmetrical femoral component or an asymmetrical femoral component, and at least a portion of the first side geometry and/or at least a portion of the second side geometry may include a reciprocal concave geometry and/or a reciprocal flat geometry. The universal tibial insert may also include at least one indicator that provides a visual notification or identification regarding whether the universal tibial insert is in the first orientation or the second orientation for purposes of proper orientation during surgery. Here, the indicator may be in the form of an alphanumeric symbol or a color-code. In another aspect of these embodiments, each of the first side geometry and the second side geometry may include a respective anterior peak terminating to a medial side of the universal tibial insert and a posterior peak terminating to a lateral side of the universal tibial insert. Here, the anterior peak may be positioned medial to the median plane, whereby the universal tibial insert may be used in a first orientation that is approximately 180 degrees relative to the second orientation.

In another embodiment as disclosed herein, a universal tibial insert may include a keyed lock plate generally symmetrical across a median plane and a frontal plane and selectively engageable with an asymmetrical tibial base plate in one of two orientations approximately 180 degrees relative to one another. Here, the keyed lock plate may include a downwardly projecting base and a pair of lock bars in spaced apart relation relative thereto, wherein the base and the lock bars cooperate with one another to form a respective pair of notches therebetween having a size and shape for select reception of at least a portion of an upwardly projecting peripheral edge of the tibial baseplate. Opposite the keyed lock plate is an upwardly presented articular surface, which may reciprocally interface with a medial condyle and a lateral condyle of a femoral component in articulatory relation therewith when the universal tibial insert is in the first orientation or in the second orientation when coupled thereto by the keyed lock plate.

In another aspect of these embodiments, one of the pair of lock bars may selectively engage an anterior tibial channel formed by an anterior overhang extending inwardly from the upwardly projecting peripheral edge thereof and the other of the pair of lock bars may selectively reside within a keyed channel of the tibial baseplate when the universal tibial insert is selectively engaged with the tibial baseplate in either of the first orientation or the second orientation. Here, each of the pair of lock bars may be of a width approximately equal to that of the keyed channel, thereby stabilizing side-to-side movement of the universal tibial insert relative to the tibial baseplate when coupled thereto in either of the first orientation or the second orientation. Each of the pair of lock bars may also include a chamfered projection forming a lock bar channel that selectively engages the anterior overhang.

Additionally, the base may also include a reciprocal pair of outwardly projecting ledges forming a pair of grooves therein that selectively engage a pair of inwardly projecting posterior overhangs on opposite sides of the keyed channel of the tibial baseplate for slotted reception therewith. The downwardly projecting base may also be of a size and shape for select reception within an inner cavity of the tibial baseplate formed by the upwardly projecting peripheral edge. Here, the keyed lock plate may include an outwardly projecting flange having a size and shape for seated reception on an upper surface of the peripheral edge of the tibial baseplate while the downwardly projecting base simultaneously seats flush within the inner cavity of the tibial baseplate.

In another alternative embodiment, the universal tibial insert may include a symmetric pair of apertures formed therein and in general alignment with a pair of bores in the tibial baseplate when in the first orientation and in the second orientation. As such, the apertures and the bores may have a size and shape for select slide through reception and tightening engagement of at least one bolt to retain the universal tibial insert to the tibial baseplate. Alternatively, an adhesive selected from the group consisting of a fibrin adhesive, collagen adhesive, polyurethane, epoxy resin, cyanoacrylates, polyesters, and zinc polycarboxylate may help couple, alone or in combination with other features disclosed herein (e.g., the aforementioned snap-fit engagement or bolt), the universal tibial insert to the tibial baseplate.

In another embodiment, the universal tibial insert may include a keyed lock plate generally symmetrical across a median plane and a frontal plane and selectively engageable with an asymmetrical tibial base plate. Here, the keyed lock plate may include a downwardly projecting base and a pair of lock bars in spaced apart relation relative thereto, which cooperate to form a groove in between for select reception and retainment of at least a portion of an upwardly projecting peripheral edge of the tibial baseplate. Moreover, an upwardly presented articular surface having a first side geometry and a second side geometry may be configured so that each interface with one of a medial condyle or a lateral condyle of a femoral component in articulatory relation relative thereto. Here, the first side geometry and the second side geometry may be symmetrical about a median plan and relatively more congruent with the medial condyle than the lateral condyle.

One aspect of these embodiments is that the first side geometry and the second side geometry may be reciprocal of one another. With respect to the congruence, a radii of the medial condyle relative to at least one radii of the first side geometry and at least one radii of the second side geometry may have a medial ratio of about 1:1 to 1:1.2 and a radii of the lateral condyle relative to at least another radii of the first side geometry and at least another radii of the second side geometry may have a lateral ratio of about 1:1.1 to 1:2. The femoral component itself may be a symmetrical femoral component or an asymmetrical femoral component, and an adhesive selected from the group consisting of a fibrin adhesive, collagen adhesive, polyurethane, epoxy resin, cyanoacrylates, polyesters, and zinc polycarboxylate may help secure the universal tibial insert to the tibial baseplate, alone or in combination with other features disclosed herein (e.g., snap-fit engagement of the universal tibial insert with the tibial baseplate). Additionally, the universal tibial insert may include at least one indicator visually identifying whether the universal tibial insert is in one of a pair of orientations relative to the tibial baseplate. Such an indicator may include an alphanumeric symbol or a color-code.

The pair of lock bars may include a width approximately equal to that of a keyed channel of the tibial baseplate and include a chamfered projection selectively engageable underneath an inwardly projecting anterior overhang of the tibial baseplate to help secure the universal tibial insert to the tibial baseplate. Moreover in this respect, the downwardly projecting base may further include a reciprocal pair of ledges that form a pair of slots therein that selectively engage a pair of inwardly projecting posterior overhangs on opposite sides of the keyed channel to facilitate further snap-fit securement thereto.

In another alternative embodiment, the downwardly projecting base may be of a size and shape for select reception within an inner cavity of the tibial baseplate and an outwardly projecting flange of the keyed lock plate may be of a size and shape for seated reception on an upper surface of the peripheral edge while the downwardly projecting base simultaneously seats flush within the inner cavity. The keyed lock plate and the upwardly presented articular surface may also couple to the tibial baseplate in a first position for use in a right total knee arthroplasty or a second position 180 degrees relative to the first position for use in a left total knee arthroplasty.

Other features and advantages of the present invention will become apparent from the following more detailed description, when taken in conjunction with the accompanying drawings, which illustrate, by way of example, the principles of the invention.

### BRIEF DESCRIPTION OF THE DRAWINGS

The accompanying drawings illustrate the invention. In such drawings:

FIG. 1 is an exploded perspective view of a total knee replacement system including a universal tibial insert as disclosed herein generally positioned between a femoral component and a tibial baseplate;

FIG. 2 is a bottom plan view of the universal tibial insert, further illustrating a base symmetric across a median plane and symmetric across a frontal plane;

FIG. 3 is bottom perspective view of the universal tibial insert, further illustrating a lock plate for coupling the universal tibial insert to the tibial baseplate;

FIG. 4 is an anterior top perspective view of the tibial baseplate symmetric across its median plane, and including a pair of posterior channels for selectively receiving a portion of the base of the universal tibial insert for snap-fit engagement therewith;

FIG. 5 is a posterior top perspective view of the tibial baseplate of FIG. 4, further illustrating an anterior channel for selectively receiving another portion of the base of the universal tibial insert therein for further snap-fit engagement therewith;

FIG. 6 is a cross-sectional view taken about the line 6-6 in FIG. 10, further illustrating a medial condyle of the femoral component generally congruent with a medial articular surface of the universal tibial insert;

FIG. 7 is a cross-sectional view taken about the line 7-7 in FIG. 10, further illustrating a lateral condyle of the femoral component relatively less congruent with a lateral articular surface of the universal tibial insert than the medial condyle with the medial articular surface as illustrated in FIG. 6;

FIG. 8 is an exploded anterior perspective view of the universal tibial insert in a right total knee replacement system;

FIG. 9 is an exploded anterior perspective view of the universal tibial insert in a left total knee replacement system;

FIG. 10 is a posterior top perspective view of the left total knee replacement system with the femoral component seated on the universal tibial insert;

FIG. 11 is an exploded perspective view of the right total knee replacement system, illustrating flexural movement of the femoral component relative to the universal tibial insert;

FIG. 12 is a top perspective view of the universal tibial insert oriented for use in the right total knee replacement system, such as illustrated in FIG. 8; and

FIG. 13 is a perspective view of the universal tibial insert oriented for use in the left total knee replacement system, such as illustrated in FIG. 9.

### DETAILED DESCRIPTION OF THE PREFERRED EMBODIMENTS

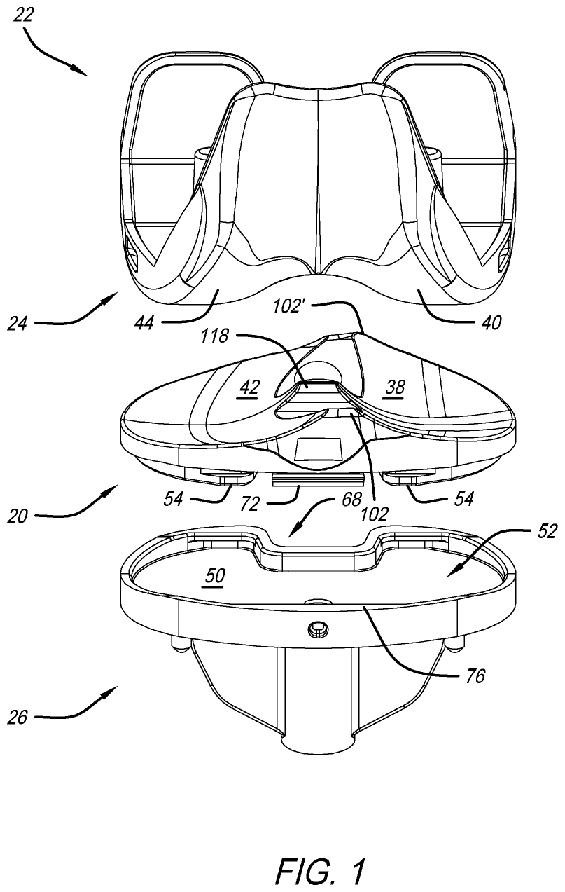

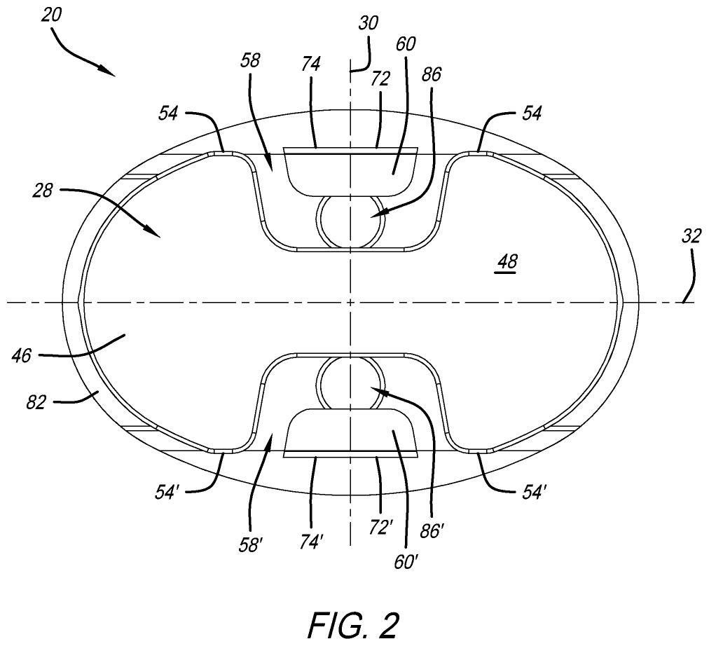

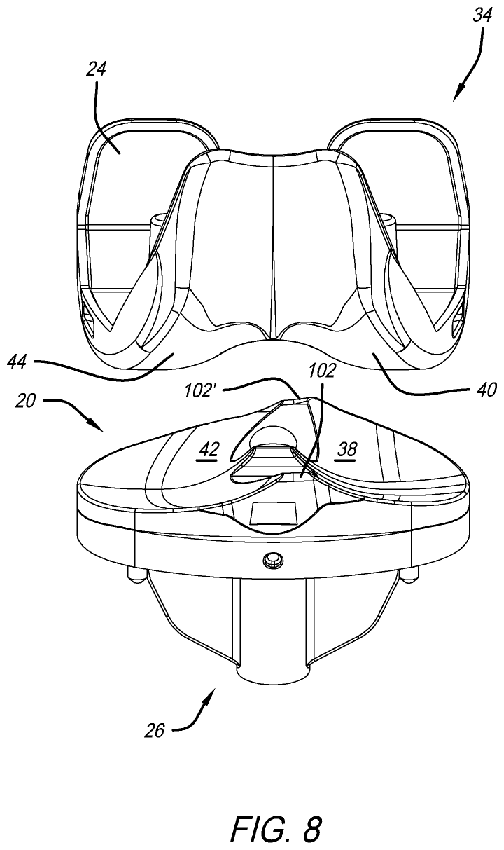

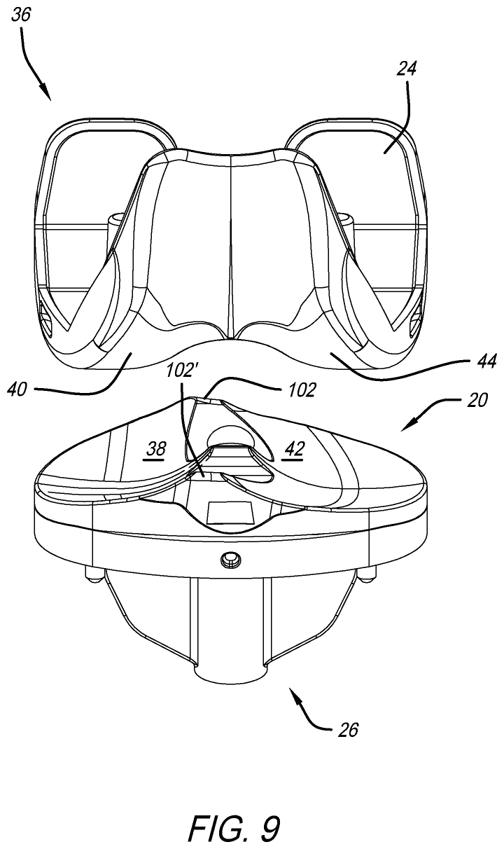

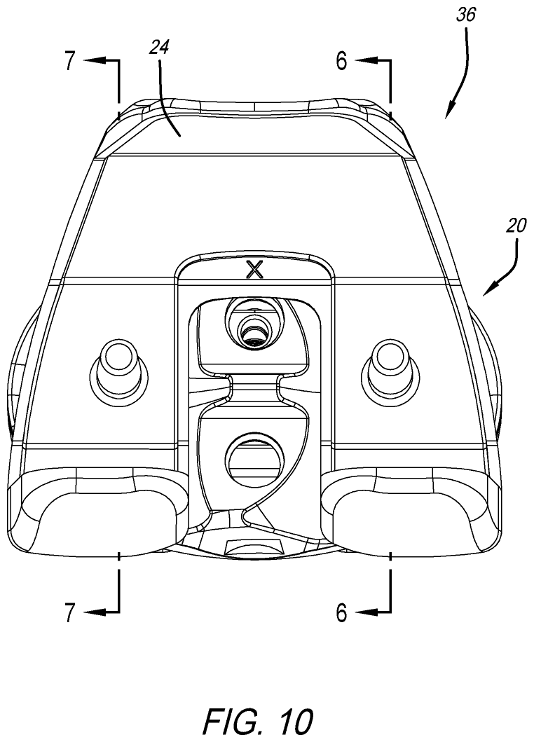

As shown in the exemplary drawings for purposes of illustration, the present invention for a universal tibial insert is generally illustrated in FIGS. 1-3 and 6-13 with respect to reference numeral 20. As illustrated in FIG. 1, the universal tibial insert 20 is for use as one of several components of a total knee replacement system 22, including for use with a femoral component 24 (including, e.g., an asymmetric femoral component or a symmetric femoral component, such as the one shown and described in U.S. Pat. No. 9,289,305, the contents of which are herein incorporated by reference in its entirety), and for coupling to a tibial baseplate 26 having a generally symmetric geometry about its median plane. Briefly, the universal tibial insert 20 as disclosed herein includes a base 28 symmetric across a median plane 30, such that its anterior and posterior sides are mirror images of one another, and symmetric across a frontal plane 32, such that its medial and lateral sides are mirror images of one another, as best illustrated in FIG. 2. As such, the universal tibial insert 20 is able to couple with the tibial baseplate 26 as part of a right total knee replacement system 34 (FIG. 8) and a left total knee replacement system 36 (FIG. 9), while at the same time consistently positioning a medial articular surface 38 designed to be relatively more congruent with a medial condyle 40 of the femoral component 24 than that of a lateral articular surface 42 designed to be less congruent with a lateral condyle 44 of the femoral component 24. As such, the universal tibial insert 20 is able to further reduce inventory required to support surgery relative to “sided” tibial inserts known in the art that can only be implanted into a right or a left knee, but not both, all while the universal tibial insert 20 remains more constrained along the medial articular surface 38 relative to the lateral articular surface 42.

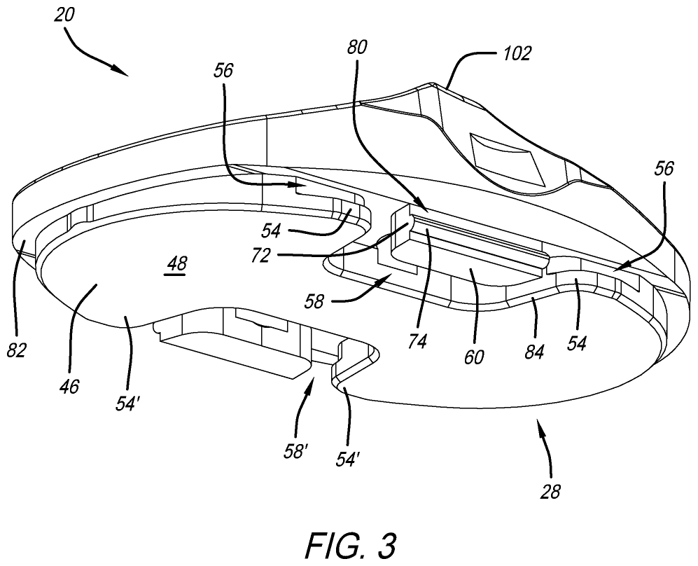

More specifically as illustrated in FIGS. 2 and 3, the base 28 includes a downwardly projecting and symmetrical keyed lock plate 46 terminating in a base surface 48 designed to sit flush against an inner surface 50 formed within a recess 52 of the tibial baseplate 26 after the universal tibial insert 20 engages with the tibial baseplate 26 in snap-fit relation. As illustrated in FIGS. 2 and 3, the symmetric lock plate 46 includes a mirrored pair of outwardly projecting ledges 54, 54′ formed below a respective pair of grooves 56, 56′ slotted within the body of the lock plate 46. Each of the pair of outwardly projecting ledges 54, 54′ are positioned on terminating sides of the lock plate 46 designed to form a respective pair of notches 58, 58′ therebetween generally enclosing a pair of downwardly projecting lock bars 60, 60′ in three-sided relation relative thereto. Accordingly, as best illustrated in FIG. 2, the base 28 of the universal tibial insert 20 is symmetrical across both the median plane 30 and the frontal plane 32. This enables the universal tibial insert 20 to couple with the tibial baseplate 26 two orientations rotated 180° relative to one another, as discussed in more detail below.

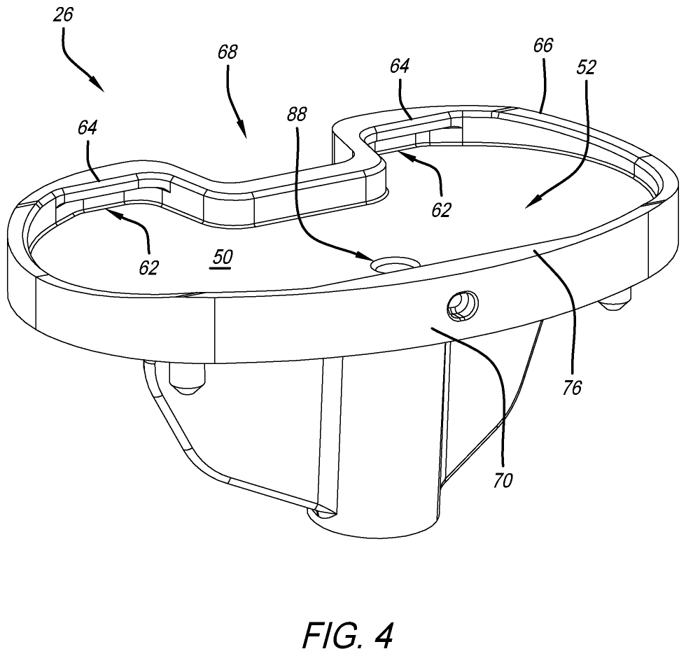

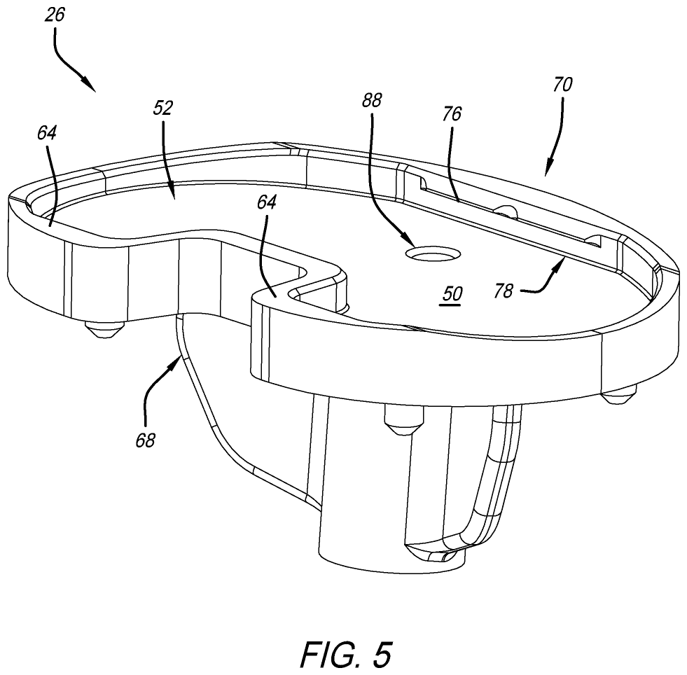

The universal tibial insert 20 may couple to the tibial baseplate 26 by first inserting one set of the pair of ledges 54 or 54′ into a corresponding pair of posterior channels 62 (best illustrated in FIG. 4) formed by a pair of posterior overhangs 64 extending out over the recess 52 from an upstanding peripheral edge 66 generally encompassing the recess 52 of the tibial baseplate 26. The pair of outwardly extending ledges 54 or 54′ may initially be inserted within the posterior channels 62 at an angle to position the respective lock bar 60 or 60′ opposite the inserted ledges 54 or 54′ into a keyed channel 68 (best illustrated in FIGS. 4 and 5) formed from the tibial baseplate 26. The keyed channel 68 helps locate the universal tibial insert 20 relative to the tibial baseplate 26 in the correct orientation for purposes of quick snap-fit engagement therewith. In this respect, once the set of the pair of ledges 54 or 54′ are at least partially inserted within the posterior channels 62, and the lock bar 60 or 60′ opposite thereof is at least partially received within the anterior channel 68, the tibial insert 20 may then be rocked down toward an anterior side 70 of the tibial baseplate 26 so that the other of the lock bar 60 or 60′ selectively snap-fit engages the anterior side 70 thereof.

Specifically in this respect, each of the lock bars 60, 60′ include an outwardly extending protrusion 72, 72′ with a leading chamfered edge 74, 74′ that contacts an anterior overhang 76 outwardly extending over the recess 52 to form an anterior channel 78 thereunder in a similar manner as the pair of posterior overhangs 64 form the respective posterior channels 62 thereunder. Through continued downward rocking movement, the respective protrusion 72 or 72′ eventually clears the anterior overhang 76 such that the respective chamfered edge 74 or 74′ is no longer in engagement therewith, whereby the respective protrusion 72 or 72′ is able to slide down underneath the anterior overhang 76 within the anterior channel 78 in snap-fit engagement therewith.

When in this position, one set of the pair of the ledges 54 or 54′ now reside within the posterior channels 62 underneath the posterior overhangs 64, the lock bar 60 or 60′ formed on the same side as the pair of ledges 54 or 54′ within the posterior channels 62 resides within the keyed channel 68, and the other of the lock bars 60 or 60′ is engaged on the anterior side 70 by way of its protrusion 72 or 72′ extending into the anterior channel 78 under the anterior overhang 76. Here, the universal tibial insert 20 is effectively in snap-fit engagement with the tibial baseplate 26. Of course, because the base 28 of the universal tibial insert 20 is symmetrical, the universal tibial insert 20 is engageable with the tibial baseplate 26 in one of two orientations that are 180° opposite each other, depending whether the universal tibial insert 20 is to be used in the right total knee replacement system 34 or the right total knee replacement system 36.

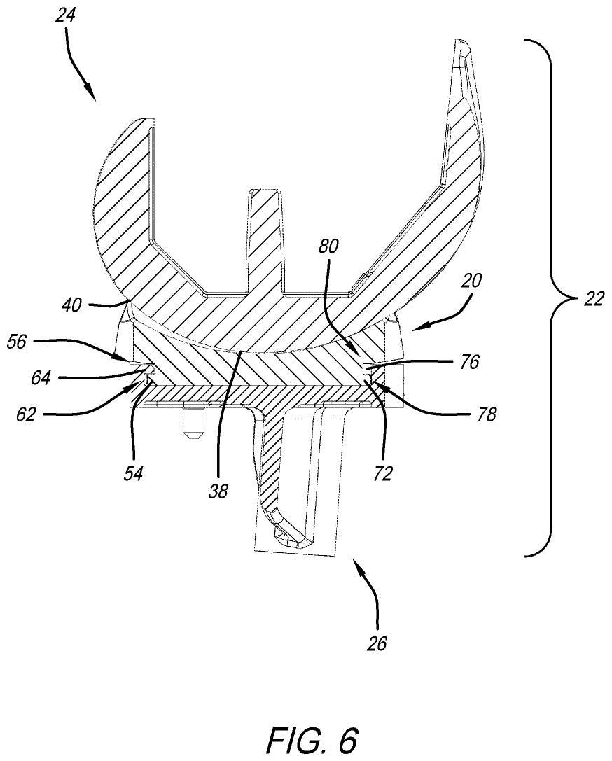

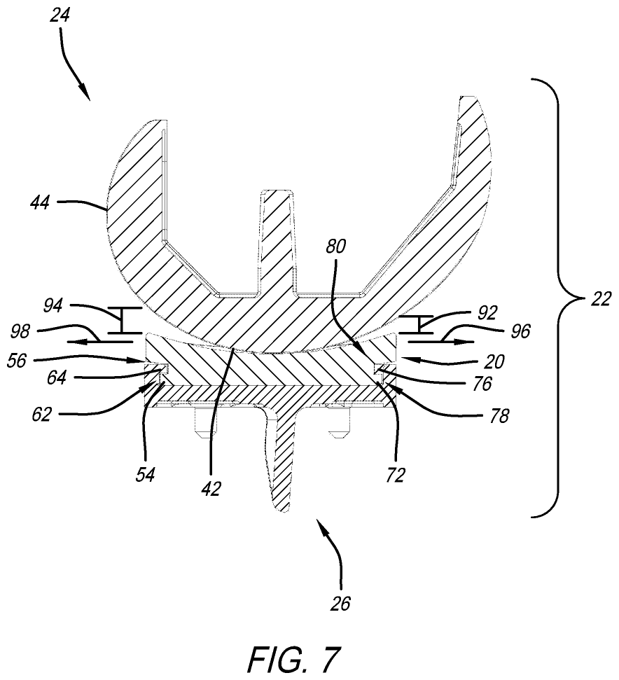

As illustrated in FIGS. 6 and 7, when the universal tibial insert 20 is snap-fit coupled to the tibial baseplate 26, the posterior overhangs 64 generally fill the entirety of the grooves 56 and the ledges 54 generally fill the entirety of the posterior channels 62, and the protrusion 72 of the lock bar 60 generally fills the entirety of the anterior channel 78. At the same time, the anterior overhang 76 engages a lock bar channel 80 (also illustrated in FIG. 3). Moreover, when the universal tibial insert 20 snap-fit couples to the tibial baseplate 26, a portion of the lock bar 60 or 60′ not otherwise engaged with the anterior channel 78 resides within the keyed channel 68 to constrain or prevent side-to-side movement of the universal tibial insert 20 relative to the tibial baseplate 26. When secured thereto, a recessed outer periphery 82 (FIG. 3) of the universal tibial insert 20 effectively formed from the downwardly extending lock plate 46 generally positioned to an interior thereof generally seats on the commensurately sized and shaped peripheral edge 66 of the tibial baseplate 26, thereby more uniformly stabilizing the universal tibial insert 20 on the tibial baseplate 26 after implantation.

Alternatively, the universal tibial insert 20 may couple to the tibial baseplate 26 by first inserting the protrusion 72 or 72′ outwardly extending from the corresponding lock bar 60 or 60′ at an angle into the anterior channel 78. At the same time, the anterior overhang 76 may at least partially extend into the lock bar channel 80 as well. The universal tibial insert 20 is then pressed downwardly into rocking engagement with the tibial baseplate 26 whereby one pair of the corresponding ledges 54 or 54′ engage with the posterior channels 62 underneath the posterior overhangs 64. The lock bar 60 or 60′ not otherwise engaged with the anterior side 70 of the tibial baseplate 26 then slides within the space formed by the keyed channel 68. The lock plate 46 may include a chamfered edge 84 around its periphery, including along each of the ledges 54, 54′, to facilitate insertion of the ledges 54 or 54′ past the posterior overhangs 64. Once therein, the posterior overhangs 64 may extend into the grooves 56 or 56′ above the corresponding ledges 54 or 54′ for snap-fit coupling thereto.

Another method for coupling the universal tibial insert 20 to the tibial baseplate 26 may include initially aligning one of the lock bars 60 or 60′ of the universal tibial insert 20 within the keyed channel 68. Then, the universal tibial insert 20 is pressed downwardly so the lock bar 60 or 60′ descends into the keyed channel 68 wherein the chamfered edge 74 of the protrusion 72 contacts the anterior overhang 76 at the same time the chamfered edges 84 along the ledges 54 or 54′ contact the posterior overhangs 64. Applying a continued downward force causes the chamfered edges 74 and 84 to somewhat deflect the posterior protrusion 72 and the ledges 54 or 54′ into the respective anterior channel 78 and the pair of posterior channels 62 for snap-fit engagement therewith, while the other lock bar 60 or 60′ is able to fully descend into the keyed channel 68. The universal tibial insert 20 is then engaged with the tibial baseplate 26 as shown and described above with respect to, e.g., FIGS. 6 and 7.

In addition to snap-fit engagement, the universal tibial insert 28 may further attach to the tibial baseplate 26 by adhesives or mechanical engagement, such as by way of a threaded screw or bolt, locking cross pin or metal clip. In one embodiment, the adhesive may be applied to the base surface 48 and/or the recessed outer periphery 82 of the universal tibial insert 20, or the adhesive may be applied to the inner surface 50 and/or the peripheral edge 66 of the tibial baseplate 26. The adhesive then serves to further retain the universal tibial insert 20 to the tibial baseplate 26 as it cures therebetween. In other embodiments, the adhesive may be used alone to exclusively retain the universal tibial insert 20 to the tibial baseplate 26. In one embodiment, the adhesive may be a bone cement (e.g., polymethylmethacrylate (PMMA) or the like) known in the art. Alternatively, the adhesive may include a fibrin adhesive, collagen adhesive, polyurethane, epoxy resin, cyanoacrylates, polyesters, or zinc polycarboxylate.

In an alternative embodiment, the universal tibial insert 20 may couple to the tibial baseplate 26 via a bolt that extends through, e.g., one of the corresponding apertures 86 or 86′ formed within the universal tibial insert 20 (FIG. 2) and aligned with a threaded bore 88 (FIGS. 4-5). Each of the apertures 86 or 86′ are symmetric to one another so that either one can align with the threaded bore 88 depending on the orientation that the universal tibial insert 20 engages the tibial baseplate 26. Here, the universal tibial insert 20 may be further locked to the tibial baseplate 26 by threading the bolt into the threaded bore 88, whereby the bolt head would apply a locking or downward pressure on the universal tibial insert 20 by way of a landing edge 90 within the corresponding aperture 86, 86′ through which the bolt threads.

As illustrated in FIGS. 8 and 9, the universal tibial insert 20 as disclosed herein may be used with the right knee total replacement system 34 (FIG. 8) and/or the left knee total replacement system 36 (FIG. 9). As briefly discussed above, the universal tibial insert 20 includes the medial articular surface 38 and the lateral articular surface 42 designed to engage the respective medial condyle 40 and the lateral condyle 44 of the femoral component 24 in articulatory relationship. While FIGS. 8-9 illustrate that the femoral component 24 has generally symmetrical condyles 40, 44, the femoral component 24 may be asymmetrical whereby the medial condyle 40 would be relatively larger than the lateral condyle 44. In either case, as illustrated best in FIGS. 6 and 7, the condyles 40, 44 are designed to be positioned within the curvatures formed by the medial articular surface 38 and the lateral articular surface 42 within the universal tibial insert 20.

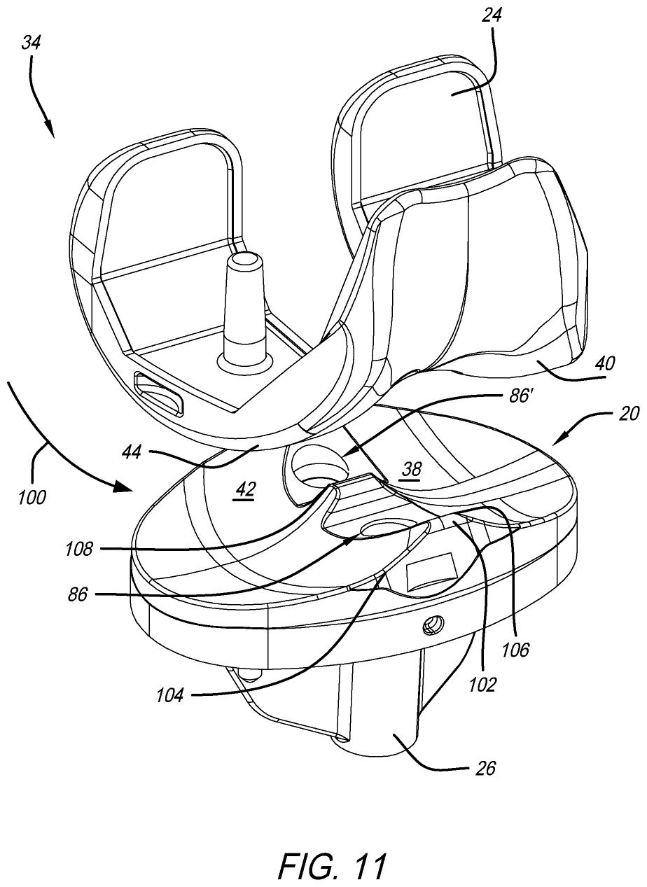

More specifically as illustrated in FIG. 6, the medial articular surface 38 is generally congruent with the medial condyle 40 on the medial side of the femoral component 24. As such, the radii of the medial condyle 40 seated on the medial articular surface 38 compared to the radii of the medial articular surface 38 is in a ratio of about 1:1 to 1:1.2. The enhanced congruence between the medial articular surface 38 and the medial condyle 40 may substantially constrain movement of the medial condyle 40 to the medial articular surface 38 during knee flexion. As illustrated in FIG. 7, when the lateral condyle 44 is seated on the lateral articular surface 42, an anterior space or gap 92 may form between the lateral articular surface 42 and the lateral condyle 44 toward the anterior of the tibial baseplate 26 and a posterior space or gap 94 may form between the lateral articular surface 42 and the lateral condyle 44 toward the posterior of the tibial baseplate 26. The radii of the lateral condyle 44 seated on the lateral articular surface 42 compared to the radii of the lateral articular surface 42 is in a ratio of about 1:1.1 to about 1:2. In an alternate embodiment, the lateral articular surface 42 may be substantially flat, thereby providing enhance movement of the lateral condyle 44 relative to the lateral articular surface 42. In this respect, the anterior gap 92 and the posterior gap 94 allow for translational movement of the lateral condyle 44 along respective directional arrows 96, 98 during knee flexion. Moreover, as illustrated in FIG. 11, because the lateral condyle 44 is not substantially constrained to the lateral articular surface 42, the lateral condyle 44 may rotate generally along directional arrow 100 about the lateral articular surface 42. The combination of constraining the medial condyle 40 to the medial articular surface 38 and allowing translational and rotational movement of the lateral condyle 44 on the lateral articular surface 42 may better approximate the normal kinematics of a healthy knee joint.

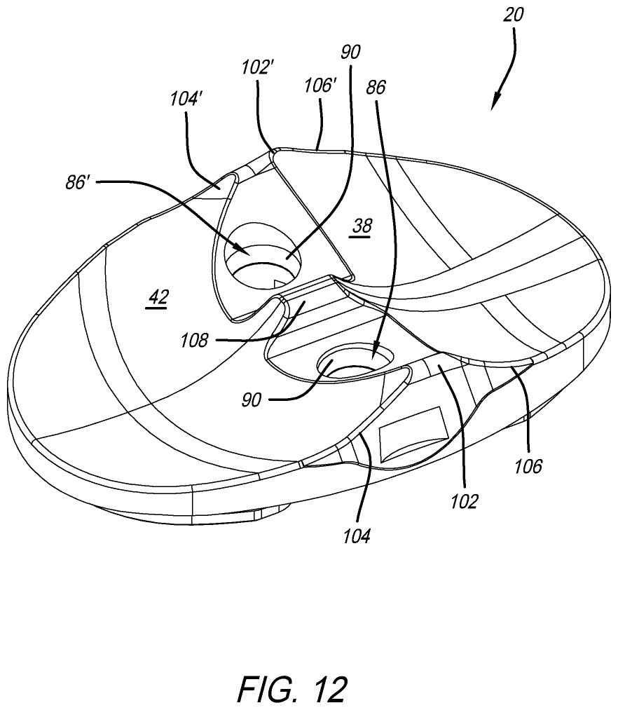

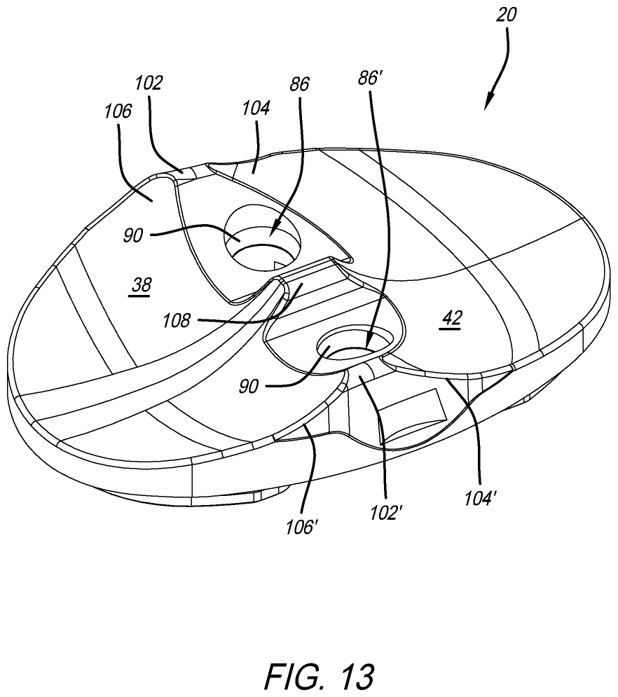

Further to constraining movement of the medial condyle 40 relative to the medial articular surface 38 and permitting relative enhanced movement of the lateral condyle 44 relative to the lateral articular surface 42, the universal tibial insert 20 includes a pair of peaks 102, 102′ culminating between a gradual slope 104, 104′ on one side and a relatively steep slope 106, 106′ on an opposite side thereof. As shown best in FIGS. 12 and 13, as a result of the difference between the respective gradual slope 104 or 104′ and the relatively steep slope 106 or 106′, the corresponding peaks 102, 102′ terminate in a position offset from the median plane of the universal tibial insert 20. For example, as illustrated in FIG. 12, the anteriorly located peak 102 terminates to a medial side and the posteriorly located peak 102′ terminates to a lateral side when used in the right total knee replacement system 34. Conversely, in FIG. 13, the anteriorly located peak 102′ terminates to a medial side while the posteriorly located peak 102 terminates to a lateral side when used in the left total knee replacement system 36. Thus, in both FIGS. 12 and 13, the anteriorly located peaks 102, 102′ are positioned medial to the median plane 30. As such, positioning the anteriorly located peaks 102, 102′ in this manner assists in constraining the medial condyle 40 to the medial articular surface 38 and helps prevent the femoral component 24 from sliding too far forward and/or off the anterior side of the universal tibial insert 20. The posteriorly located peak 102 or 102′ assists in constraining the lateral condyle 44 to prevent the femoral component 24 from sliding too far backward and/or off the posterior side of the universal tibial insert 20. As such, these features further effectively constrain the femoral component 24 to provide the designed knee kinematics.

Moreover, the universal tibial insert 20 may further include an upwardly slopped and centrally located post 108 that may assist in aligning the femoral component 24 with the universal tibial insert 20. For example, the post 108 may assist in providing medial-to-lateral stability for the femoral component 24 and, as a secondary consideration, the centrally located post 108 may further assist in rolling back the femoral component 24 to a generally parallel position with the universal tibial insert 20 during flexion.

Lastly, the medial articular surface 38 and/or the lateral articular surface 42 may include an indicator that may assist the surgeon in coupling the universal tibial insert 20 to the tibial baseplate 26 in the correct orientation. For example, the medial articular surface 38 and/or lateral articular surface 42 may include an alphanumeric symbol such as a “M” on the medial articular surface 38 to designate the same and/or a “L” on the lateral articular surface 42 to designate the same. Of course, the indicator may include any symbol, alphanumeric or otherwise, to assist in differentiating the medial articular surface 38 from the lateral articular surface 42. Alternatively, the indicator may be color-coded to differentiate the medial articular surface 38 from the lateral articular surface 42. For example, in one embodiment, the medial articular surface 38 may be blue and the lateral articular surface 42 may be green. Moreover, the universal tibial insert 20 may be made of high-density plastic such as polyethylene or the like.

Although several embodiments have been described in detail for purposes of illustration, various modifications may be made without departing from the scope and spirit of the invention. Accordingly, the invention is not to be limited, except as by the appended claims.

What is claimed is:

1. A universal tibial insert, comprising:
a base selectively engageable with a tibial baseplate; and
an upwardly presented articular surface having a first side geometry and a second side geometry configured to interface with one of a medial condyle or a lateral condyle of a femoral component in articulatory relation thereof, the articular surface being symmetrical about a median plane such that the first side geometry and the second side geometry are relatively more congruent with the medial condyle and relatively less congruent with the lateral condyle when in either one of a first orientation for use in one of a right total knee arthroplasty or a left total knee arthroplasty or a second orientation for use in the other of the right total knee arthroplasty or the left total knee arthroplasty.

2. The universal tibial insert of claim 1, wherein the first side geometry and the second side geometry are reciprocal such that the first side geometry and the second side geometry have approximately the same congruence with the medial condyle and the lateral condyle.

3. The universal tibial insert of claim 2, wherein a radii of the medial condyle of the femoral component relative to a radii of the first side geometry or a radii of the second side geometry comprises a medial ratio of about 1:1 to 1:1.2.

4. The universal tibial insert of claim 2, wherein a radii of the lateral condyle of the femoral component relative to a radii of the first side geometry or a radii of the second side geometry comprises a lateral ratio of about 1:1.1 to 1:2.

5. The universal tibial insert of claim 1, wherein the femoral component comprises a symmetrical femoral component or an asymmetrical femoral component.

6. The universal tibial insert of claim 1, wherein at least a portion of the first side geometry and at least a portion of the second side geometry comprises a reciprocal concave geometry and a reciprocal flat geometry.

7. The universal tibial insert of claim 1, wherein the universal tibial insert includes at least one indicator visually identifying whether the universal tibial insert is in the first orientation or the second orientation.

8. The universal tibial insert of claim 7, wherein the indicator comprises an alphanumeric symbol or a color-code.

9. The universal tibial insert of claim 1, wherein each of the first side geometry and the second side geometry include a respective anterior peak terminating to a medial side of the universal tibial insert and a posterior peak terminating to a lateral side of the universal tibial insert.

10. The universal tibial insert of claim 9, wherein the anterior peak is positioned medial to the median plane.

11. The universal tibial insert of claim 1, wherein the first orientation is approximately 180 degrees relative to the second orientation.

12. A universal tibial insert, comprising:
a keyed lock plate generally symmetrical across a median plane and a frontal plane and selectively engageable with an asymmetrical tibial base plate in a first orientation and a second orientation approximately 180 degrees relative to the first orientation; and
an upwardly presented articular surface configured to reciprocally interface with a medial condyle and a lateral condyle of a femoral component in articulatory relation therewith when the universal tibial insert is in the first orientation or in the second orientation.

13. The universal tibial insert of claim 12, wherein the keyed lock plate includes a downwardly projecting base and a pair of lock bars in spaced apart relation relative thereto, the base and the lock bars cooperate to form a respective pair of notches therebetween having a size and shape for select reception of at least a portion of an upwardly projecting peripheral edge of the tibial baseplate.

14. The universal tibial insert of claim 13, wherein one of the pair of lock bars selectively engages an anterior tibial channel formed by an anterior overhang extending inwardly from the upwardly projecting peripheral edge thereof and the other of the pair of lock bars selectively resides within a keyed channel of the tibial baseplate when the universal tibial insert is selectively engaged with the tibial baseplate in either of the first orientation or the second orientation.

15. The universal tibial insert of claim 14, wherein the pair of lock bars comprise a width approximately equal to that of the keyed channel, thereby stabilizing side-to-side movement of the universal tibial insert relative to the tibial baseplate when coupled thereto in either of the first orientation or the second orientation.

16. The universal tibial insert of claim 14, wherein each of the pair of lock bars include a chamfered projection forming a lock bar channel that selectively engages the anterior overhang.

17. The universal tibial insert of claim 14, wherein the base includes a reciprocal pair of outwardly projecting ledges forming a pair of grooves therein that selectively engage a pair of inwardly projecting posterior overhangs on opposite sides of the keyed channel of the tibial baseplate for slotted reception therewith.

18. The universal tibial insert of claim 13, wherein the downwardly projecting base is of a size and shape for select reception within an inner cavity of the tibial baseplate formed by the upwardly projecting peripheral edge.

19. The universal tibial insert of claim 18, wherein the keyed lock plate includes an outwardly projecting flange having a size and shape for seated reception on an upper surface of the peripheral edge of the tibial baseplate while the downwardly projecting base simultaneously seats flush within the inner cavity of the tibial baseplate.

20. The universal tibial insert of claim 12, wherein the universal tibial insert includes a symmetric pair of apertures formed therein and in general alignment with a pair of bores in the tibial baseplate when in the first orientation and in the second orientation, the apertures and the bores having a size and shape for select slide through reception and tightening of at least one bolt therein to retain the universal tibial insert to the tibial baseplate.

21. The universal tibial insert of claim 12, including an adhesive selected from the group consisting of a fibrin adhesive, collagen adhesive, polyurethane, epoxy resin, cyanoacrylates, polyesters, and zinc polycarboxylate coupling the universal tibial insert to the tibial baseplate.

22. A universal tibial insert, comprising:
a keyed lock plate generally symmetrical across a median plane and a frontal plane and selectively engageable with an asymmetrical tibial base plate, the keyed lock plate including a downwardly projecting base and a pair of lock bars in spaced apart relation relative thereto cooperating to form a groove in between for select reception and retainment of at least a portion of an upwardly projecting peripheral edge of the tibial baseplate; and
an upwardly presented articular surface having a first side geometry and a second side geometry each configured to interface with one of a medial condyle or a lateral condyle of a femoral component in articulatory relation, the first side geometry and the second side geometry being symmetrical about a median plan and relatively more congruent with the medial condyle than the lateral condyle.

23. The universal tibial insert of claim 22, wherein the first side geometry and the second side geometry are reciprocal of one another.

24. The universal tibial insert of claim 22, wherein a radii of the medial condyle relative to at least one radii of the first side geometry and at least one radii of the second side geometry comprises a medial ratio of about 1:1 to 1:1.2.

25. The universal tibial insert of claim 24, wherein a radii of the lateral condyle relative to at least another radii of the first side geometry and at least another radii of the second side geometry comprises a lateral ratio of about 1:1.1 to 1:2.

26. The universal tibial insert of claim 22, wherein the femoral component comprises a symmetrical femoral component and an adhesive selected from the group consisting of a fibrin adhesive, collagen adhesive, polyurethane, epoxy resin, cyanoacrylates, polyesters, and zinc polycarboxylate secures the universal tibial insert to the tibial baseplate.

27. The universal tibial insert of claim 22, wherein the universal tibial insert includes at least one indicator visually identifying whether the universal tibial insert is in one of a pair of orientations relative to the tibial baseplate.

28. The universal tibial insert of claim 27, wherein the indicator comprises an alphanumeric symbol or a color-code.

29. The universal tibial insert of claim 22, wherein the pair of lock bars comprise a width approximately equal to that of a keyed channel of the tibial baseplate and include a chamfered projection selectively engageable underneath an inwardly projecting anterior overhang of the tibial baseplate.

30. The universal tibial insert of claim 29, wherein the downwardly projecting base includes a reciprocal pair of ledges forming a pair of slots therein that selectively engage a pair of inwardly projecting posterior overhangs on opposite sides of the keyed channel.

31. The universal tibial insert of claim 22, wherein the downwardly projecting base is of a size and shape for select reception within an inner cavity of the tibial baseplate and an outwardly projecting flange of the keyed lock plate is of a size and shape for seated reception on an upper surface of the peripheral edge while the downwardly projecting base simultaneously seats flush within the inner cavity.

32. The universal tibial insert of claim 22, therein the keyed lock plate and the upwardly presented articular surface couple to the tibial baseplate in a first position for use in a right total knee arthroplasty or a second position 180 degrees relative to the first position for use in a left total knee arthroplasty.
